(19) United States
(12) Patent Application Publication
(10) Pub. No.: US 2023/0031881 A1
(43) Pub. Date: Feb. 2, 2023

(54) A SWAY BED

(71) Applicant: SWAY SLEEP SYSTEMS PTY LTD, Elwood (AU)

(72) Inventors: Andrew MACKAY, (US); Jacob ALDRICH, (US)

(21) Appl. No.: 17/754,873

(22) PCT Filed: Oct. 23, 2020
(86) PCT No.: PCT/AU2020/051142

§ 371 Date: Apr. 14, 2022

(30) Foreign Application Priority Data
Oct. 23, 2019 (AU) 2019903993

(51) Int. Cl. A47C 21/00

(52) U.S. Cl. CPC A47C 21/006

(57) ABSTRACT

A sway bed has a fixed base and a swaying frame defining a bed surface movably attached thereto by a plurality of sway linkages. An oscillating drive operably interfaces the fixed base and the swaying frame. The oscillating drive comprises a geared motor turning a distal end of a crank slidably retained within a channel substantially orthogonal with respect to the bed surface.

### FIELD OF THE INVENTION

This invention relates generally to rocking and/or swaying beds such as for sleep assistance.

### BACKGROUND OF THE INVENTION

Rocking and/or swaying beds have been proposed for sleep assistance and of which various configurations exist including U.S. Pat. No. 4,958,391 A (Byron) which discloses a sway bed comprising a sway frame suspended from chains of a base frame.

U.S. Pat. No. 7,234,179 B2 (Chun-Fa) discloses a rocking assembly for a bed which has a mattress frame, a base frame and a rocking driver. The base frame supports the mattress frame. The rocking driver is mounted on the base frame and has a motor, a flywheel and a connecting rod. The flywheel is rotated by the motor. The connecting rod is connected eccentrically to the flywheel and pivotally to the mattress frame. When the motor turns, the connecting rod rocks the mattress frame so a person lying on the bed rocks from side to side and pressure on individual parts of the person's body is alleviated so blood can circulate to the areas and bedsores will not develop.

U.S. Pat. No. 3,737,924 A (Davis) discloses a hemicylindrical bed driven by the movement of an arm for controlled movement and user security.

U.S. Pat. No. 2,808,828 A (Salem) discloses a bed having an upper mattress supporting frame which is capable of universal tilting movement relative to a lower supporting base.

U.S. Pat. No. 3,439,363 A (Meeks) discloses a rocking bed with a frame within which a cradle with rocker members at each end is mounted with means for producing a rocking motion of the cradle.

CN 2783857 Y (Meng) discloses a sway bed having a sway frame movably coupled to a base frame by way of a plurality of sway linkages. Meng further discloses a drive operative between the sway frame and the base frame. The drive comprises a piston eccentrically coupled to a motor driveshaft at a proximal end and acting on a lever at a distal end. A distal end of the lever is coupled to the sway frame by a connector rod such that the motor moves the sway frame side to side.

US 2007/0094792 A1 (Sims) discloses a sway bed wherein a sway frame is suspended from swing arms of a base frame. A crank turned by a motor acts on a connector rod to swing the sway frame side to side.

CN 1515212 A (Jiang) discloses a sway bed comprising a sway frame held atop pivoted props. The sway frame has a fixing bracket to which a connector rod turned by a crank is connected to move the sway frame side to side.

The present invention seeks to provide a sway bed, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided herein a sway bed comprising a fixed base and a swaying frame defining a bed surface movably attached thereto by a plurality of sway linkages and an oscillating drive operably interfacing the fixed base and the swaying frame, wherein the oscillating drive comprises a geared motor turning a distal end of a crank slidably retained within a channel substantially orthogonal with respect to the bed surface.

Trial and experimentation found that this particular configuration provided less jerky motion as compared to crank type arrangements such as those taught by Chun-Fa, Sims, Meng and Jiang.

The geared motor may be fixed with respect to the base and the channel may be fixed with respect to the swaying frame and a bracket that widens towards the swaying frame for lateral structural resilience under load may define the channel.

Each sway linkage may comprise a sway arm and a fixed arm, the fixed arm fixed orthogonally to the swaying frame at an upper end thereof and the sway arm pivotally coupled to the fixed base at an upper end thereof at an upper pivot point and to the fixed arm at a lower end thereof at a lower pivot point, the fixed arm extending above the upper pivot point.

The crank may rotate about a rotational axis aligned with a longitudinal axis of the bed such that the swaying frame sways sideways with respect to the fixed base and wherein, in end elevation cross section, the fixed base may comprise a pair of upper pivot points for a pair of respective sway linkages and wherein the channel may be located between the pair of sway linkages.

These sway linkages stably support the sway frame as the sway frame moves side to side in each pair of sideways sway linkages may support the swaying frame either side of the channel under load therebetween.

At a bottom dead centre position, the channel may be located substantially equidistantly between the upper pivot points so that the swaying frame is laterally balanced.

The fixed arms may connect to the sway frame at connection points and the connection points may be further apart than the upper pivot points so that the swaying frame moves in a slightly concave trajectory.

In side elevation cross section, the channel may be closely adjacent the upper pivot points, such as less than 20 cm or preferably less than 10 cm apart, to prevent torsion between the swaying frame and the fixed base.

The fixed base may comprise a sideways beam defining the pair of upper pivot points therealong and bed may comprise a plurality of sway linkages in longitudinal alignment along longitudinal axes either side of the channel.

For larger sized beds, the electric motor may turns a pair of cranks synchronously, the cranks rotating about a rotational axis aligned with a longitudinal axis of the bed, the cranks having distal ends slidably retained within respective channels, the channels spaced along the longitudinal axis. A centrepoint between the channels coincides substantially with a longitudinal centrepoint along the longitudinal axis of the swaying frame. This particular arrangement reduces or eliminates portion between the swaying frame and the fixed base.

The distal end may comprise bushing and the bushing may have flat sides tightly interfacing inner sides of the channel to reduce or eliminate hysteresis.

The channel may comprise a non-metallic peripheral insert for interfacing the bushing.

The fixed base may comprise major side coverings affixed thereto and the swaying frame may comprise minor side coverings affixed thereunderneath and wherein the side coverings move vertically adjacently without forming a substantial gap therebetween in vertical elevation therebetween to conceal access to the interior working of the bed. The minor side coverings may move on inner sides of the major side coverings.

The sway bed may comprise a modular unit comprising the oscillating drive and the sway linkages, the modular unit comprising a pair of lower sideways beams which removably affix at distal ends thereof to longitudinal beams of the fixed frame and pair of upper sideways beams at least one of the fixing to and forming part of the swaying frame. As such, the modular unit may be easily removed for maintenance.

The channel may be located at a centrepoint of the swaying frame along a longitudinal axis of the swaying frame to prevent torsion between the fixed frame in the swaying frame.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a large-size variant of a sway bed in accordance with an embodiment;

FIG. 2 shows a smaller size variant of a sway bed in accordance with an embodiment;

FIG. 3 shows a modular unit for the large-size variant comprising an oscillating drive and sway linkages in accordance with an embodiment;

FIG. 4 shows a modular unit for the small-size variant;

FIGS. 5 and 6 illustrate motion of the large-size variant;

FIGS. 7 and 8 illustrate motion of the small-size variant;

FIG. 9 shows an exploded perspective representation of the small-size variant; and

FIG. 10 shows an exploded perspective representation of the large-size variant.

### DESCRIPTION OF EMBODIMENTS

A sway bed 100 comprises a fixed base 101 and a swaying frame 102 movably coupled thereto by a plurality of sway linkages 104 and an oscillating drive operably interfacing the fixed base 101 and the swaying frame 102.

The swaying frame defines a bed surface 103.

Longitudinal and derivatives thereof will be described herein with reference to a longitudinal (i.e. head-to-toe) axis 105 and sideways, lateral and derivatives thereof will be described herein with reference to a sideways axis 106.

The swaying frame 102 may comprise one or more longitudinal beams 106 and a plurality of sideways beams 107. Parallel sideways planks 108 may lie atop the beams 106, 107.

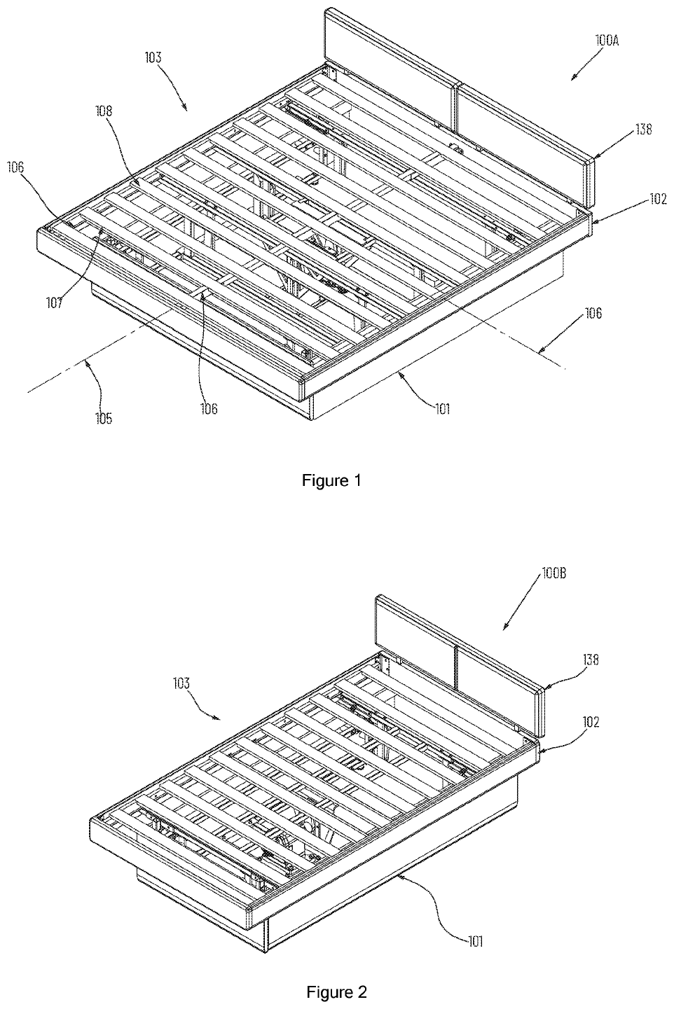

FIG. 1 shows a larger variant 100A (such as a king or queen size bed) as compared to a smaller variant 100B of FIG. 2 (such as a single bed).

The oscillating drive 105 comprises a geared motor 109 turning a distal end 110 of a crank 111 slidably retained within a channel 112 substantially orthogonal with respect to the bed surface 103. The geared motor 109 may produce more than 100 N m for the small size variant 1008 and produce more than 200 N m for the large-size variant 100A. The geared motor 109 may comprise gearing that does not slip back when load is applied to the swaying frame 102. For example, the geared motor 109 may comprise a worm drive such that, if the motor 109 comes to rest away from a bottom dead centre position, the swaying frame 102 does not fall back down.

FIG. 4 and five show wherein the geared motor 109 may be provided as a modular unit wherein the geared motor 109 may be fixed to the base 101 by a motor mounting framework 113 which may comprise a mounting plate 114, sideways beams 115 and spaced apart right angle attachment brackets 116 having fastener apertures 117 therethrough for fastening to corresponding longitudinal beams of the fixed base 101.

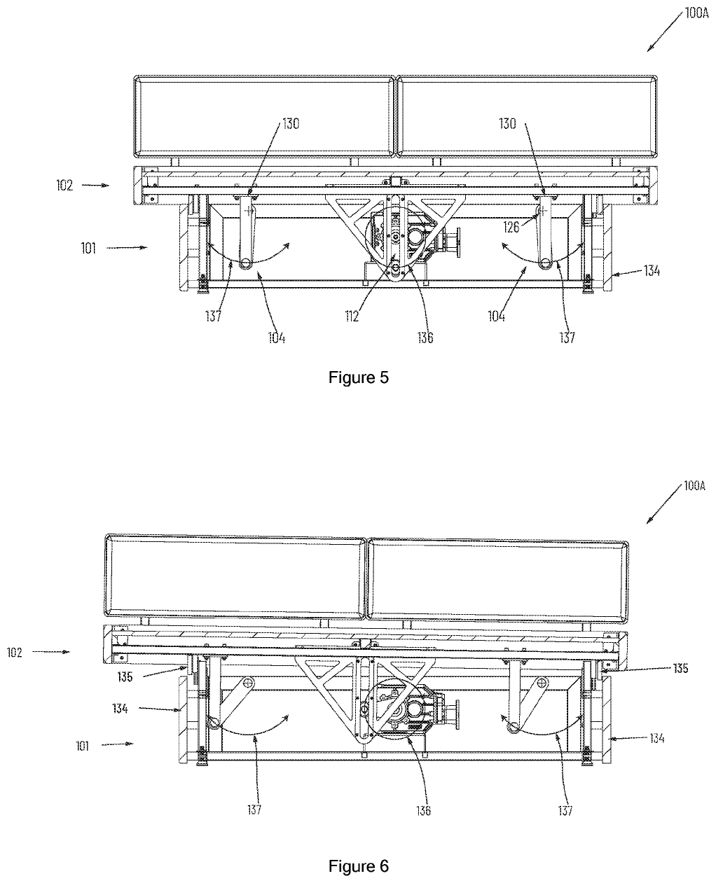

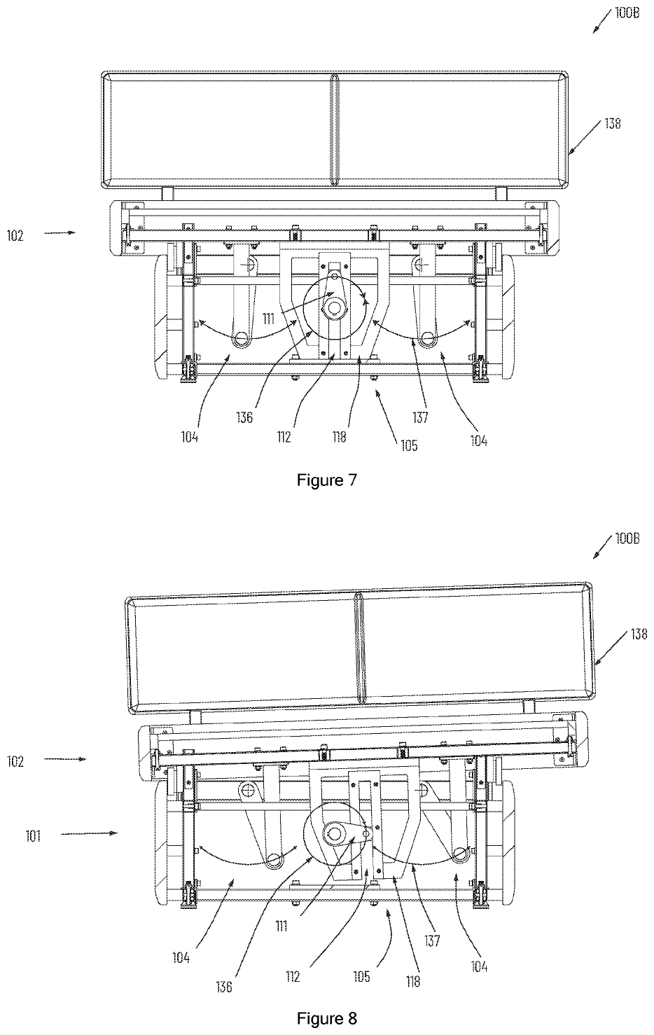

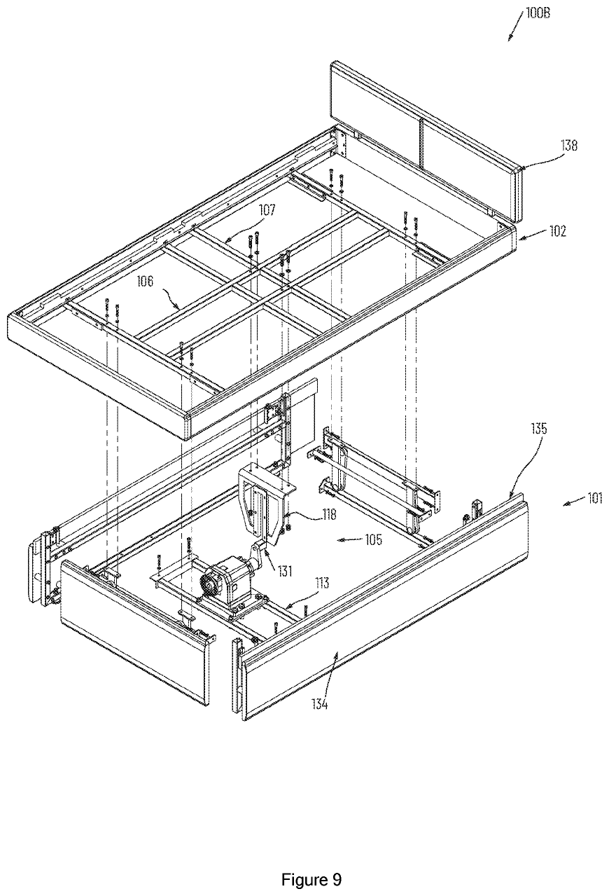

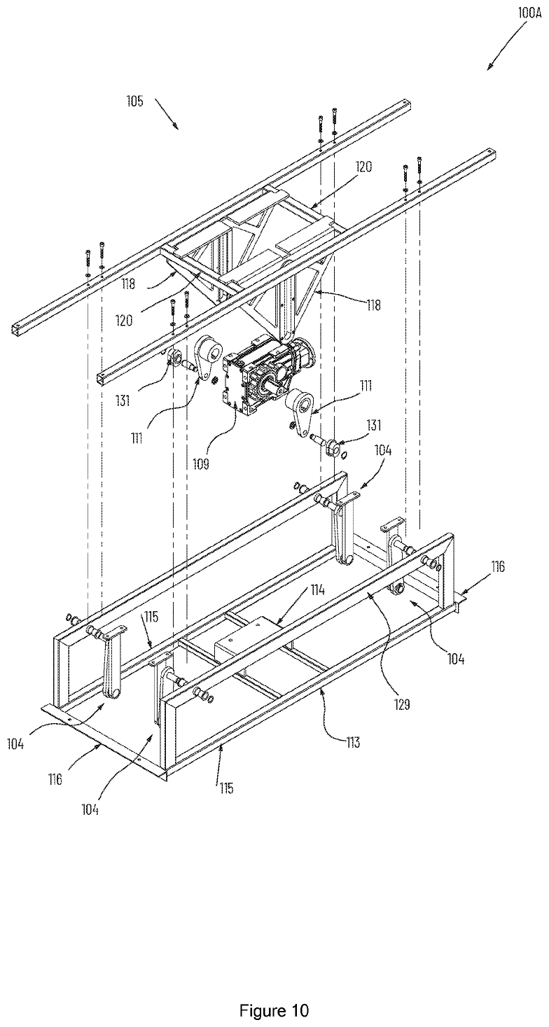

As is also shown in FIGS. 4-5, the channel 112 may be fixed with respect to the swaying frame 102. In this regard, the bed 100 may comprise a bracket 118 extending beneath the swaying frame 102 and wherein the bracket 118 defines the channel 112. The bracket 112 may widen towards the swaying frame 102 for lateral structural resilience when under load.

In the embodiment shown, the bracket 118 may be substantially triangular defining a nadir 123 and the channel 112 may extend substantially almost the entire height of the bracket 118.

The bracket 118 may be pressed from a metal plate to form an orthogonal wide edge flange 119 which may engage over longitudinal suspension bars 120 and punched to form outer struts 121 and substantially orthogonal inner struts 122.

Each sway linkage 104 may comprise a sway arm 124 and a fixed arm 125. The fixed arm 125 is fixed to the swaying frame 102 at an upper end thereof. The sway arm 124 may be pivotally coupled to the fixed base 101 at an upper end thereof at an upper pivot point 126 and to the fixed arm 125 at a lower end thereof at a lower pivot point 127. The fixed arm 125 may extend above the upper pivot point 126.

The sway linkage 104 may comprise tight tolerance bearings to prevent twisting between the fixed arm 125 and the sway arm 124 to minimise or eliminate torsion between the fixed base 101 and the swaying frame 102 when under load. In this regard, the fixed arm 125 and the sway arm 124 may be planar and lie flat sides adjacent each other to allow close together pivotal coupling to minimise the effect of the sway linkage to minimise torsional effect.

As the distal end 110 of the crank 111 travels in a circular path 136, the lower pivot points 127 travel in an arc.

The crank 111 may rotate about a rotational axis 128 aligned with the longitudinal axis 105 of the bed 100 such that the swaying frame 102 sways sideways with respect to the fixed base 101.

As shown in FIG. 5, in end elevation cross-sectional, the fixed base 101 may comprise a pair of upper pivot points 126 for a pair of respective sway linkages 104 and wherein the channel 112 is between the upper pivot points 126. The fixed base 101 may comprise sideways beams 129 each supporting the pair of upper pivot points 126. The channel 112 may be located substantially equidistantly between the upper pivot points 126.

As is shown in FIG. 5, the fixed arms 124 may connect to the swaying frame 102 at a pair of connection points 130. The connection points 130 may be the same distance apart as the upper pivot points 126 such that the swaying frame 102 is maintained horizontal as it sways side-to-side. However, in alternative embodiments, the connection points 130 may be further apart than the pair of the upper pivot points 126 so that the sway frame 102 sways in a slightly concave arc as illustrated in FIGS. 6 and 8 wherein at each sideways oscillation extremity, an outer side edge of the sway frame 102 is slightly higher than an inner side edge of the sway frame 102. In embodiments, the positioning of the upper pivot points 126 may be adjustable to control the sway arc of the swaying frame 102. In one embodiment the bed 100 comprises a worm drive for each upper pivot point 130 wherein a rotational actuator rotates threaded rods thereof to dynamically move the upper pivot points 130 further apart or closer together.

In the embodiments shown, the swaying frame 102 sways side-to-side (i.e. laterally). However, in an alternative embodiment, the swaying frame 102 may sway along the longitudinal axis 105 using the same sway linkage 104 arrangement. In this regard, the upper connection points 130 thereof may be either the same spacing as the respective upper pivot points 126 such that the swaying frame 102 remained horizontal while swaying or alternatively wherein the upper pivot points 126 are closer together so that the sway frame 102 sways in a slightly concave arc as described above.

In side elevation cross-section, the channel 112 is preferably closely adjacent the upper pivot points 126, such as less than 20 cm apart, preferably less than 10 cm apart.

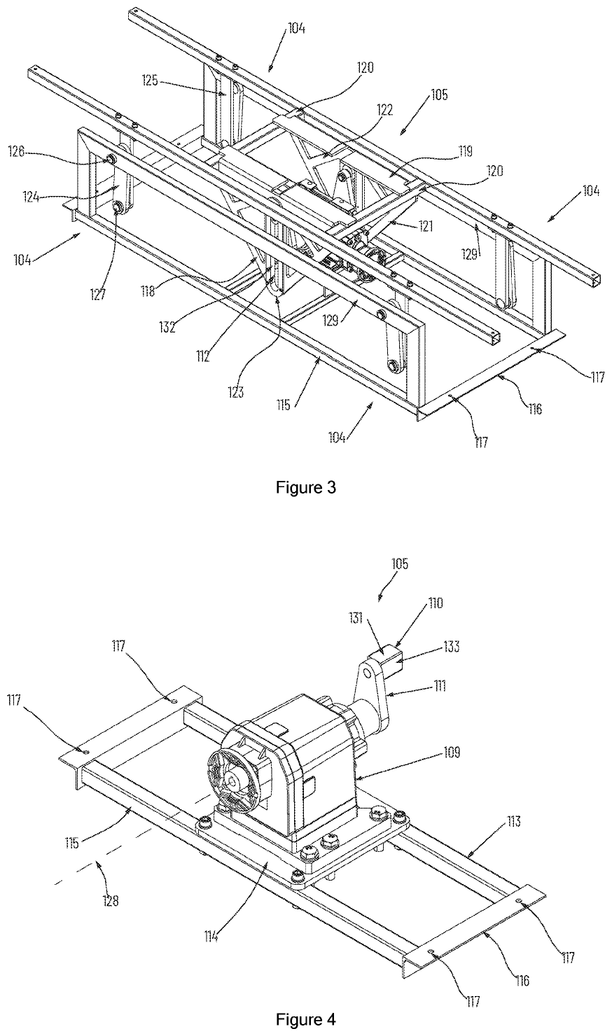

The geared motor 109 of the oscillating drive 105 of the larger variant 100A shown in FIG. 3 may turn a pair of cranks 111 synchronously either side thereof about the rotational axis 128 aligned with the longitudinal axis 105 of the bed 100, the cranks 111 have respective distal ends 110 slidably retained within respective channels 112.

A centrepoint between the channels 112 may coincide substantially with a longitudinal centrepoint along the longitudinal axis 105 of the swaying frame 102.

The oscillating drive 105 of the smaller variant 100B shown in FIG. 4 may turn a single crack 111. The distal end 110 may similarly coincide with a centrepoint along the longitudinal axis 105 of the swaying frame 102.

The distal end 110 of the crank 111 may comprise a bushing 131 such of high-density plastic. The bracket 118 may further comprise a plastic interface and insert 132 defining a non-metallic edge interfacing the plastic bushing 131.

The bushing 131 may comprise planar sides 133 lying flat against respective inner straight edges of the channel 112. The planar sides 133 may fit within the channel 112 with tight tolerance (that is of less than 1 mm) to reduce or eliminate hysteresis.

The fixed base 101 may comprise major side covering 134 affixed thereto and the swaying frame 102 may comprise minor side coverings 135 affixed there underneath. The side coverings 134, 135 move vertically adjacently without forming a gap in vertical elevation therebetween, thereby not accessibly concealing the interior of the bed 100. The minor side coverings 135 may slide on inner sides of the major side coverings 134.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

The term “approximately” or similar as used herein should be construed as being within 10% of the value stated unless otherwise indicated.

1. A sway bed comprising a fixed base and a swaying frame defining a bed surface movably attached thereto by a plurality of sway linkages and an oscillating drive operably interfacing the fixed base and the swaying frame, wherein the oscillating drive comprises a geared motor turning a distal end of a crank slidably retained substantially orthogonal with respect to the bed surface, wherein each sway linkage comprises a sway arm and a fixed arm, the fixed arm fixed orthogonally to the swaying frame at an upper end of the fixed arm and the sway arm pivotally coupled to the fixed base at an upper end of the sway arm at an upper pivot point and to the fixed arm at a lower end thereof at a lower pivot point, the fixed arm extending above the upper pivot point.

2. The sway bed as claimed in claim 21, wherein the geared motor is fixed with respect to the base and the channel is fixed with respect to the swaying frame.

3. The sway bed as claimed in claim 2, further comprising a bracket extending beneath the swaying frame, wherein the bracket defines the channel.

4. The sway bed as claimed in claim 3, wherein the bracket widens towards the swaying frame.

5. (canceled)

6. The sway bed as claimed in claim 1, wherein the crank rotates about a rotational axis aligned with a longitudinal axis of the bed such that the swaying frame sways sideways with respect to the fixed base and wherein, in end elevation cross section, the fixed base comprises a pair of upper pivot points for a pair of respective sway linkages and wherein the distal end of the crank is retained between the pair of sway linkages.

7. The sway bed as claimed in claim 6, wherein the fixed arms connect to the sway frame at a pair of respective connection points and wherein the pair of connection points are further apart than the pair of upper pivot points.

8. The sway bed as claimed in claim 6, wherein, at a bottom dead centre position, the distal end of the crank is retained substantially equidistantly between the upper pivot points.

9. The sway bed as claimed in claim 6, wherein, in side elevation cross section, the distal end of the crank is retained is closely adjacent the upper pivot points.

10. The sway bed as claimed in claim 9, wherein closely adjacent is less than 20 cm.

11. The sway bed as claimed in claim 6, wherein the fixed base comprises a sideways beam supporting the pair of upper pivot points therealong.

12. The sway bed as claimed in claim 6, wherein the bed comprises a plurality of sway linkages in longitudinal alignment along longitudinal axes either side of the distal end of the crank.

13. The sway bed as claimed in claim 1, wherein the geared motor turns a pair of cranks synchronously, the cranks rotating about a rotational axis aligned with a longitudinal axis of the bed, the cranks having distal ends each slidably retained substantially orthogonally with respect to the bed surface and being spaced along the longitudinal axis.

14. The sway bed as claimed in claim 14, wherein a centrepoint between the distal ends coincides substantially with a longitudinal centrepoint along the longitudinal axis of the swaying frame.

15. The sway bed as claimed in claim 21, wherein the distal end comprises bushing.

16. The sway bed as claimed in claim 15, wherein the bushing has flat sides interfacing inner sides of the channel.

17. The sway bed as claimed in claim 16, wherein the channel comprises a non-metallic peripheral insert for interfacing the bushing.

18. The sway bed as claimed in claim 12, wherein the fixed base comprises major side coverings affixed thereto and wherein the swaying frame comprises minor side coverings affixed thereunderneath and wherein the side coverings move vertically adjacently without forming a substantial gap therebetween in vertical elevation.

19. The sway bed as claimed in claim 1, wherein the sway bed comprises a modular unit comprising the oscillating drive, the modular unit comprising a pair of lower sideways beams which removably affix at distal ends thereof to longitudinal beams of the fixed frame and pair of upper sideways beams at least one of the fixing to and forming part of the swaying frame.

20. The sway bed as claimed in claim 1, wherein the distal end of the crank is slidably retained at a centrepoint of the swaying frame along a longitudinal axis of the swaying frame.

21. The sway bed as claimed in claim 1, wherein the distal end of the crank is slidably retained within a channel substantially orthogonal with respect to the bed surface.
